(19) United States
(12) Patent Application Publication
(10) Pub. No.: US 2023/0263745 A1
(43) Pub. Date: Aug. 24, 2023

(54) SYSTEM, METHOD AND COMPUTER PROGRAM FOR PROVIDING AN ASSESSMENT OF A MEDICAL RISK, METHOD FOR OBTAINING A MODEL FOR THE SYSTEM, METHOD FOR ASSESSING A MEDICAL RISK AND NUTRITIONAL SUPPLEMENTS

(71) Applicants: SORBONNE UNIVERSITE, Paris (FR); ASSISTANCE PUBLIQUE-HOPITAUX DE PARIS, Paris (FR)

(72) Inventors: Antonin LAMAZIERE, Paris (FR); Charlotte DUPONT, Bagneux (FR); Rachel LEVY, Paris (FR); Guillaume BACHELOT, Villeneuve Les Avignon (FR)

(21) Appl. No.: 18/003,900

(22) PCT Filed: Jul. 3, 2020
(86) PCT No.: PCT/FR2020/051185

§ 371 Date: Dec. 29, 2022

(51) Int. Cl. A61K 31/07; G16H 50/30; A61K 31/047; A61K 31/015; A61P 15/08

(52) U.S. Cl. CPC A61K 31/07; A61K 31/015; A61K 31/047; A61P 15/08; G16H 50/30

(57) ABSTRACT

An automated system (100) for processing physiological parameter measurements provides an assessment of a medical risk. The system includes a model (106) previously built by machine learning to provide an idiopathic infertility score (S) of a couple which is to be assessed and is formed of a male and a female, from received measurements (p1... p13) of the physiological parameters. The physiological parameters include at least one physiological parameter for the male and at least one physiological parameter for the female.

The present invention relates to the field of the infertility and more particularly to the idiopathic infertility. More particularly, the present invention relates to system, method and computer program for providing an assessment of a medical risk, a method for assessing a medical risk and nutritional supplements.

The infertility, which affects both male and female, is a recurring problem in our societies, as the couples find it increasingly difficult to have children. Our lifestyles, the environment, the fact that we have children late in life, may be the beginning of an explanation. More and more couples are seeking help for this problem. It is estimated that around 15% of couples of childbearing age will one day face infertility.

The causes of infertility can be related to problems of male or female origin or both. In this case, the diagnosis is carried out through analysis of the sperm (spermogram-spermocytogram) and a female assessment (ovarian assessment and imaging); the detection of abnormalities allow then to explain the infertility problems of the couple. The male infertility is usually characterised by an alteration of the sperm parameters (oligoasthenoteratozoospermia) or an alteration of the sperm quality (fragmentation of the sperm DNA). In female, the most common causes are dysovulations (polycystic ovary syndrome (PCOS), which affects between 5 and 10% of women), ovarian insufficiency (reduced ovarian reserve) and the absence of ovulation (referred to as anovulation). Other conditions include tubal infertility and uterine cavity anomalies. In male, the causes, apart from sperm abnormalities detected by a spermogram, may also be due to erectile dysfunction.

Approximately one third of infertility cases are of female origin, one third of male origin and are the result of the abnormalities described above. However, one third of infertility cases remain unexplained, with no biological or physiological cause for the failure to conceive by the couple. The spermogram is normal or subnormal, the female assessment is not abnormal and the couple has regular sexual intercourse without contraception. This is known as idiopathic infertility, or infertility with unknown causes.

Depending on the causes of female or male infertility, treatments may be recommended. The medically assisted reproduction (MAP) is often recommended for male and female infertility. There are also drug (hormones) and surgical treatments that can improve the fertility of the couples.

Alternatives to medical, surgical or invasive treatments (MAP) have also been described in the literature, aiming to allow the couples to find the right time for allowing the female to start a pregnancy. In the patent application WO2019057561, the subject of the invention is an electronic system in the form of a bracelet capable of capturing various physiological parameters, mainly temperature but also heart rate and respiratory rate, in order to detect events related to a pregnancy of a female, such as the ovulation, the conception and the miscarriage. Said bracelet has a sensor system in contact with the skin to measure one or more physiological parameters. A processor is configured to receive a user input indicating a duration of actual menstruations and determine time windows, in order to analyse physiological parameters of the female, using the duration of actual menstruations. The processor is further configured to detect the pregnancy-related events by comparing the physiological parameters determined and recorded for a first time window with those determined and recorded for a second time window, to indicate the pregnancy-related events when defined detection criteria are met, and to use the user input for pregnancy-related events to optimise the detection of these events with automated learning algorithms.

The weakness of this type of electronic system is that it is based on a limited number of parameters and measuring mainly temperature, respiratory rate or heart rate does not seem to allow to provide a relevant understanding of the complexity of the infertility.

Another approach for trying to help the patients with infertility problems is the one for example described in the application WO2019060882. The invention relates to a clinical decision support system in the field of the infertility which consists of a computer system configured to support the clinical decision making associated with the treatment of patients during an ovarian stimulation cycle. The system comprises one or more computing devices programmed to receive patient-specific data for a plurality of patients; create at least one decision model (e.g. a regression model) using the patient data; receive patient input data for at least one patient; provide the input data of the patient as input to the at least one decision model; obtain the output of the decision model; and generate at least one treatment recommendation of the patient for presentation via a user interface based on the output of the decision model. Although interesting, this invention is limited to the treatments implemented during IVF based on previous treatments, it does not allow to characterise the potential causes and the ways of treating the idiopathic infertility problems outside IVF.

Thus, there is a need for assessing the risk of idiopathic infertility, as well as a need for solutions to understand and treat the idiopathic infertility.

An automated system for treating physiological parameter measurements for providing an assessment of a medical risk is therefore proposed, characterised in that it comprises a model previously constructed by machine learning to provide an idiopathic infertility score for a couple to be assessed, formed of a male and a female, on the basis of measurements received of physiological parameters, and in that these physiological parameters comprise at least one physiological parameter of the male and at least one physiological parameter of the female.

Surprisingly, the inventors have shown that by looking at the problem holistically and considering the fertility not as separate male and female individuals but as a couple, it is possible, based on the analysis of physiological parameters (biological and non-biological variables), to characterise the idiopathic infertility and also to propose therapeutic options to the couples without resorting to IVF.

Optionally, the model comprises a linear combination of the measurements of the physiological parameters.

Optionally also, the physiological parameters comprise at least one of: an anthropometric parameter, a metabolic parameter and a parameter of the oxidative status.

Optionally also, the physiological parameters also comprise the following variables:
Serum retinol levels in female;
Blood glucose in male and female;
Visceral fat content in female and male;
BMI for female and male;
Waist measurements for female and male;
Serum beta-carotene levels in male;
Serum lutein levels in female; and
Serum alpha carotene levels in male and female.

Optionally also, the physiological parameters also comprise:
HDL in male;
Hip measurement of female and male;
CO exp of male;
Serum selenium levels in female;
Serum beta carotene levels in female;
Serum lutein levels in male;
Testosterone in male;
DHEA in male;
Cortisol in male; and
Androstenedione in male.

Optionally also, the physiological parameters also comprise:
Serum lutein levels in female;
Serum alpha carotene levels in female and male;
Weight of female and male;
HDL in female;
Testosterone in male;
16OHP of male;
Serum retinol levels in male;
Corticosterone in male
21DB of male;
11 BOH4 of male;
17OHP of male;
Serum cobalamin levels in female and male;
Age of the female and the male;
Cortisone in male
21DF of male
11DFof male;
Serum alpha tocopherol levels in female and male;
DOC of male;
Triglycerides in male and female;
17OH Pregn of male;
Serum folate levels in female and male;
DHT in male;
Creatinine in male and female;
Pregn of male;
Serum lycopene levels in female and male;
Serum vitamin C levels in female;
Glutathione in male and female;
Vitamin D for female;
Systolic blood pressure in female and male;
Cholesterol in female and male;
LDL in female and male;
Progesterone in male;
Ferritin in female and male;
AMH in male;
Serum zinc levels in male and female;
Beta cryptoxanthin in female;
Diastolic blood pressure in female and male
Serum vitamin D levels in male;
Aldosterone in male;
Height of the male and the female;
Glutathione Peroxidase in male;
Selenium in male; and
Vitamin C in male.

Optionally also, the automated system also comprises:
associations associating treatments with respective zones of a multidimensional space formed by the physiological parameters; and
a module for selecting the treatment associated with the zone in which the measurements of the physiological parameters of the couple to be assessed are located.

Also proposed is a method for obtaining a model for an automated system according to the invention, comprising:
for each of several couples in a cohort, obtaining physiological parameter measurements from a male of the couple and a female of the couple and a fertility status of that couple, with the fertility status of at least one of the couples indicating that the couple is idiopathic infertile and the fertility status of at least one other of the couples indicating that this couple is fertile;
selecting all the physiological parameters obtained; and
determining a model, comprising:
   training a model by a training algorithm, based on measurements of the selected physiological parameters of the couples in the cohort, so that the trained model provides a fertility score predicting the fertility status of the couple, with the training algorithm providing an importance of each physiological parameter in the prediction,
   assessing the model to validate or invalidate it,
   if the model is validated, determining an importance of each physiological parameter in the prediction and selecting some of the physiological parameters according to their importance, and then repeating the determination step for a model with the newly selected physiological parameters,
   if the model is invalidated, providing the last validated model.

Optionally, the method further comprises, prior to the step of determining a model, identifying at least one couple whose measurements are statistically aberrant and removing each identified couple from the cohort.

Also proposed is a method for the automated treating of physiological parameter measurements for providing an assessment of a medical risk of idiopathic infertility, characterised in that it comprises the use of a model previously constructed by machine learning to provide an infertility score for a couple to be assessed, formed by a male and a female, on the basis of received physiological parameter measurements, and in that these physiological parameters comprise at least one physiological parameter of the male and at least one physiological parameter of the female.

Optionally, the model comprises a linear combination of the measurements of the physiological parameters.

Optionally also, the physiological parameters comprise at least one of: an anthropometric parameter, a metabolic parameter and a parameter of the oxidative status.

Optionally also, zones of a multidimensional space formed by the physiological parameters are respectively associated with treatments and furthermore comprising the selection of the treatment associated with the zone in which the measurements of the physiological parameters of the couple to be assessed are located.

Also proposed is a computer program downloadable from a communication network and/or recorded on a computer-readable medium, characterised in that it comprises instructions for executing a method according to the invention, when said computer program is executed on a computer.

Also proposed is a method for assessing a medical risk of idiopathic infertility for a couple, comprising the steps of:
collecting, for a couple consisting of a male and a female, at least one physiological parameter of the male and at least one physiological parameter of the female;
calculating an infertility score by inputting these parameters into an automated system for treating physiological parameter measurements according to the invention; and
comparing this score with a threshold, if this score is below the threshold, the couple will be assessed as being at risk of idiopathic infertility.

A nutritional composition is also proposed comprising at least one modulator (agonist or antagonist) allowing for correcting a deficiency or an excess of a selected physiological parameter for the last validated model in the model obtaining method according to the invention.

Also proposed is a nutritional composition comprising at least one nutrient, a measurement of which forms a selected physiological parameter for the last model validated in the model obtaining method according to the invention.

Optionally, the nutritional composition according to the invention may be for use in the treatment of the idiopathic infertility.

Also proposed is a nutritional composition comprising at least one of the compounds selected from vitamin A (retinol), alpha-carotene, beta-carotene and lutein and a physiologically acceptable carrier, in particular for its use in treating the idiopathic infertility.

Optionally, the nutritional composition comprises at least two, preferably at least three, more preferably the four compounds.

Optionally also, the nutritional composition comprises at least one for at least two or the three] of the compounds selected from vitamin A, alpha-carotene and lutein and a physiologically acceptable carrier for its use in treating the idiopathic infertility in a female.

Also proposed is a nutritional composition comprising at least one for both] of the compounds selected from alpha-carotene and beta-carotene and a physiologically acceptable carrier for its use in treating the idiopathic infertility in a male.

The invention will be better understood with the aid of the following description, given only by way of example and made with reference to the attached drawings in which:

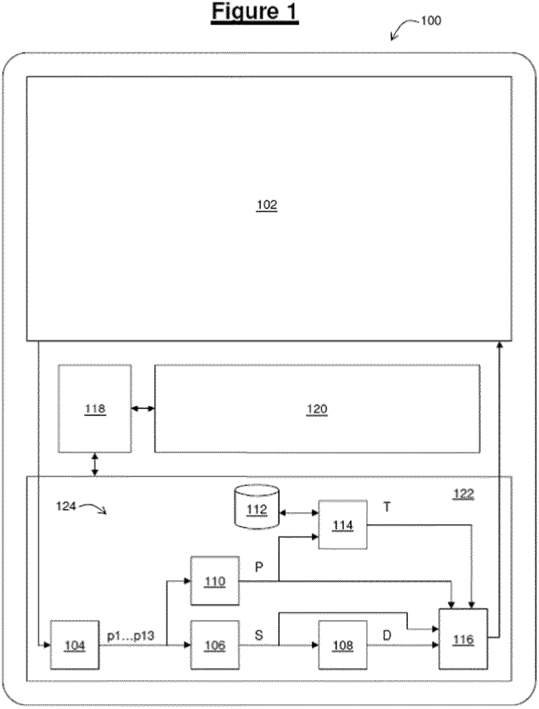

FIG. 1 is a simplified view of an automated system according to the invention for treating data for the provision of an assessment of a medical risk of idiopathic infertility,

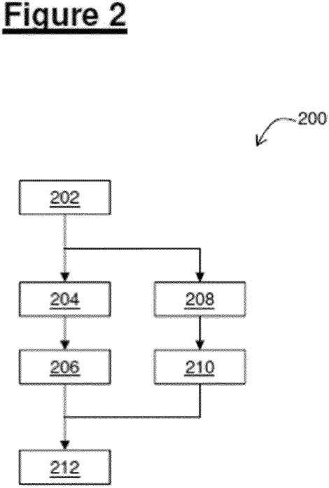

FIG. 2 is a block diagram illustrating the steps of a method according to the invention of treating data for the provision of an assessment of a medical risk of idiopathic infertility,

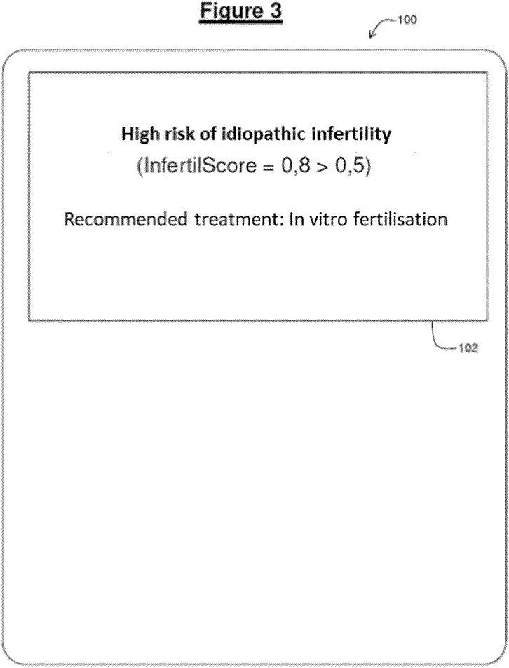

FIG. 3 is a view of the system in FIG. 1 displaying information about the assessment conducted,

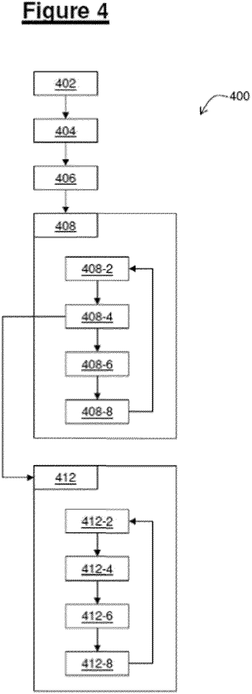

FIG. 4 is a block diagram illustrating the steps in a method for obtaining a model for the system of FIG. 1,

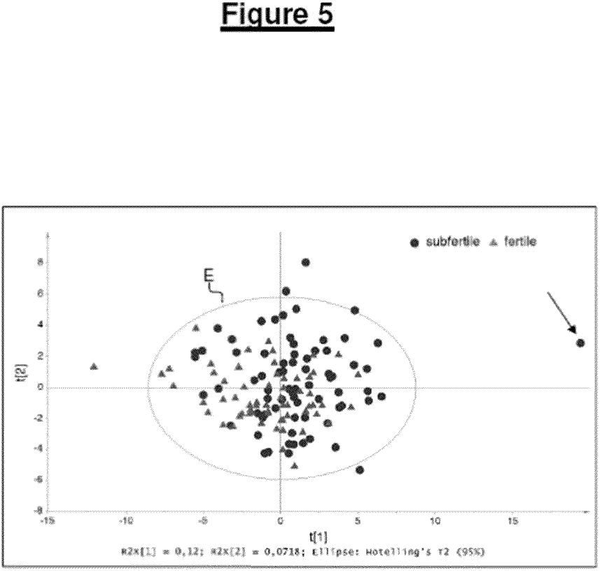

FIG. 5 shows the result of a principal component analysis (PCA) on a developmental cohort of idiopathic fertile and infertile couples, for 80 physiological parameters measured on these couples,

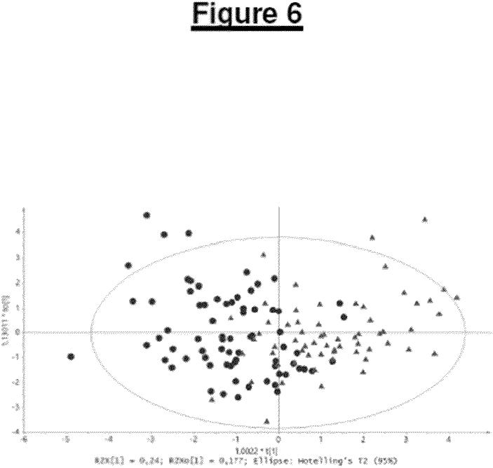

FIG. 6 is a score plot resulting from an Orthogonal Partial Least Squares - Discriminant Analysis (OPLS-DA) carried out on the development cohort, for thirteen physiological parameters measured on these couples,

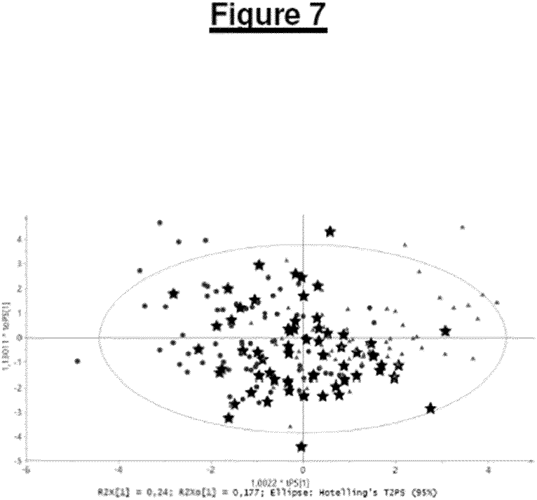

FIG. 7 repeats FIG. 6, adding the couples from a test cohort, without differentiating between fertile and idiopathic infertile couples,

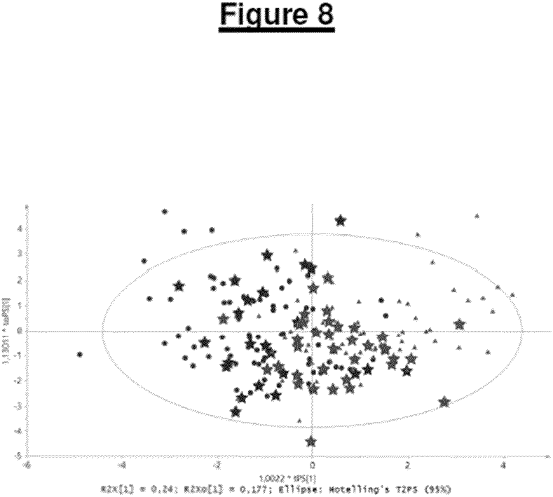

FIG. 8 is identical to FIG. 7, except that fertile and infertile idiopathic couples are differentiated,

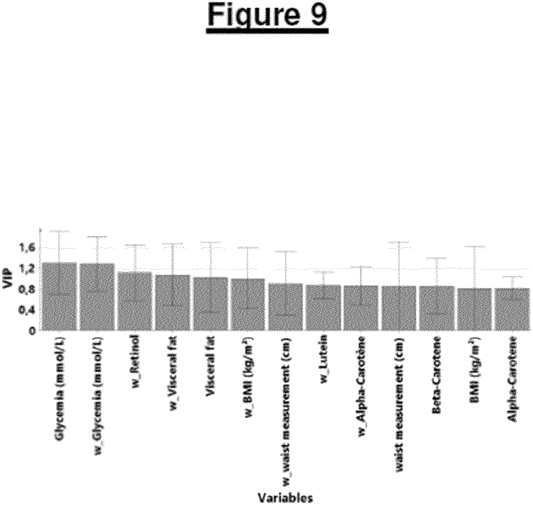

FIG. 9 is a bar chart showing the Variable Importance in Projection (VIP) of the thirteen physiological parameters, these VIP were obtained from the scope of the OPLS-DA analysis of the developmental cohort,

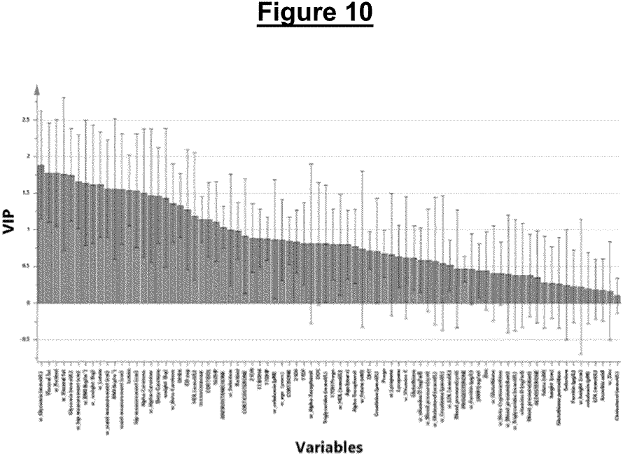

FIG. 10 is a bar chart showing the VIP of eighty physiological parameters, in the scope of an OPLS-DA analysis carried out on the total cohort (developmental cohort and test cohort), for these eighty physiological parameters,

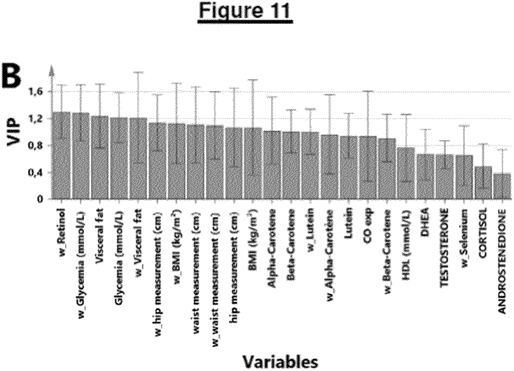

FIG. 11 is a bar chart showing the VIP for twenty-four of the eighty physiological parameters in FIG. 10, in the scope of an OPLS-DA analysis carried out on the total cohort (developmental cohort and test cohort), for these twenty-four physiological parameters,

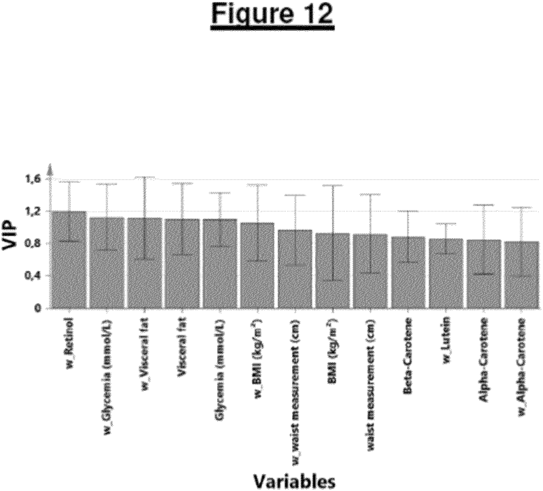

FIG. 12 is a bar chart showing the VIP of thirteen selected physiological parameters selected from the twenty-four in FIG. 11, in the scope of an OPLS-DA analysis of the total cohort (developmental cohort and test cohort), for these thirteen physiological parameters,

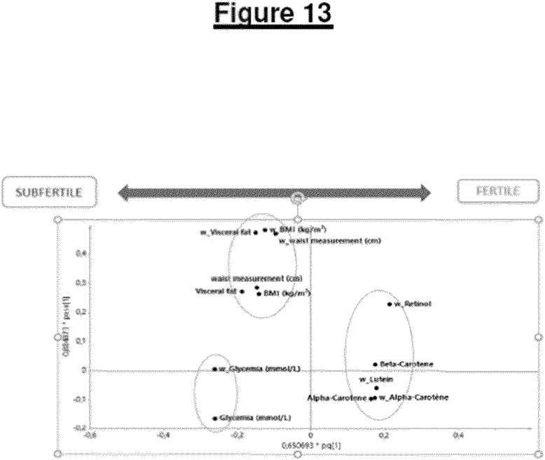

FIG. 13 is a loadings plot of the OPLS-DA analysis carried out on the development cohort, for the thirteen physiological parameters,

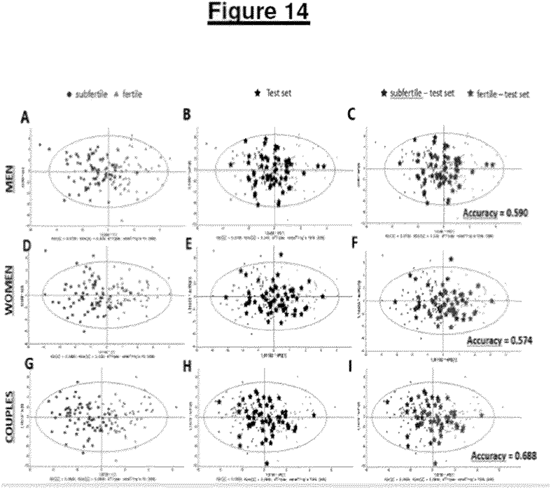

FIG. 14 shows an assembly of score plots resulting from OPLS-DA analysis on the development cohort, illustrating the differences in results between the use of male and female physiological parameters, male-only physiological parameters and female-only physiological parameters, adding the couples from a test cohort, without differentiating between fertile and idiopathic infertile couples at first and then differentiating between them.

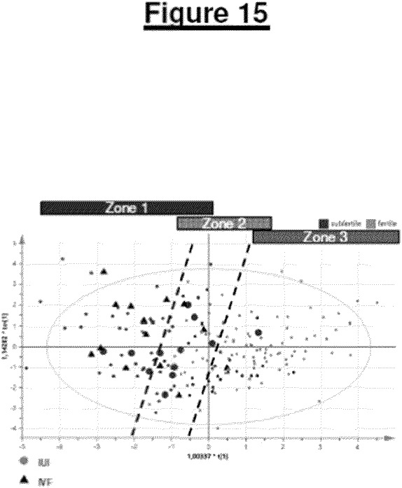

FIG. 15 is a score plot resulting from an Orthogonal Partial Least Squares - Discriminant Analysis (OPLS-DA) carried out on the total cohort (development cohort and test cohort), for the thirteen physiological parameters, indicating the treatments (IUI: Intrauterine insemination, and IVF: in vitro fertilisation) having succeeded (pregnancy), and

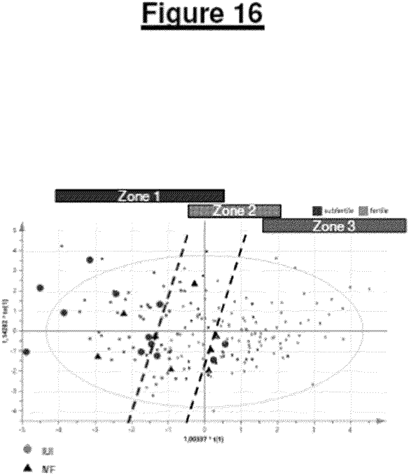

FIG. 16 is a graph similar to FIG. 15, except that it shows which treatments did not work (no pregnancy).

With reference to FIG. 1, an example 100 of automated system according to the invention of data treating will now be described. More specifically, as will be apparent below, the system 100 is designed to automatically treat physiological parameter measurements for the provision of an assessment of a medical risk of idiopathic infertility of a couple referred to as to be assessed, consisting of a male and a female. Thus, the system 100 provides a clinical decision assistance, for example for the practitioner.

The system 100 firstly comprises a human/machine interface 102 comprising at least one information input device and at least one information presentation device, for example a visual presentation device such as a screen. In the example described, the human/machine interface 102 comprises a touch screen acting as both the information input device and the information presentation device. For example, the system 100 is a computer tablet or a smartphone.

The system 100 further comprises a data receiving module 104, designed to receive measurements of the predefined physiological parameters of the couple to be assessed and to provide them to the other elements of the system 100. For example, the data receiving module 104 is designed to cooperate with the human/machine interface 102 to receive the measurements. For example, a user of the system 100 may enter the measurements by hand via the human/machine interface 102. Alternatively, some or all of the measurements can be received from a remote device (not shown), such as a computer, via a wireless or wired connection.

In addition, the receiving module 104 may be designed to receive, for example from a remote device (not shown), such as a computer, via a wireless or wired connection, an update.

The physiological parameters comprise at least one physiological parameter of the male and at least one physiological parameter of the female.

The physiological parameters that can be taken into account for the implementation of the method according to the invention are in particular chosen from among: age, anthropometric parameters, parameters of the oxidative status and metabolic parameters.

Anthropometric parameters include:
the arterial pressure (referred to as “blood pressure (syst)” and “blood pressure (diast)” in the figures): the systolic and diastolic blood pressures are preferably measured with a blood pressure cuff at the level of each of the arms of the patient after a 5-minute rest in the lying position; the value taken is the average of the two measurements;
the height in cm (referred to as “height (cm)” in the figures);
the weight in kg (referred to as “weight (kg)” in the figures); the weight and the height can be assessed by an apparatus Tanita BC-420MA analyser;
the visceral fat content (referred to as “visceral fat” in the figures) measured by impedance measurement (TANITA);
the waist measurement (referred to as “waist measurement” in the figures) measured at the narrowest point between the lower edge of the ribs and the iliac crest;
the hip measurement (referred to as the “hip measurement” in the figures) measured, for example, with a sewing tape;
the Body Mass Index (referred to as “BMI” in the figures) which is calculated by dividing the weight (in kg) by the square of the height (m²).

Among the parameters of the oxidative status, including micronutrients, it is included:
folate (vitamin B9) and cobalamin (vitamin B12), for example measured by radioisotope assay;
glutathione, determined for example by enzymatic and spectrometric methods;
serum zinc levels, which can be measured by flame atomic absorption spectrometry (a suitable apparatus is the model 3110; Perkin Elmer, Norwalk, CT);
serum selenium level which can be measured as described by Czernichow 2009 Am J Clin Nutr or Akbaraly, 2007 Journal of gerontology;
the serum level of vitamin C, for example, measured after blood is taken into assays tubes containing heparin and the plasma is centrifuged and recovered; 0.5 mL of plasma is then diluted one-tenth in 4.5 mL of a 5% aqueous solution of metaphosphoric acid and frozen. The serum level of vitamin C is then determined using high performance liquid chromatography coupled to a UV detector (HPLC-UV);
serum levels of retinol (vitamin A), alpha-tocopherol (vitamin E), alpha-carotene, beta-carotene, lycopene, lutein, zeaxanthin, beta-cryptoxanthin which can be measured by HPLC-UV from blood samples; and
Gluthatione peroxidase activity which can be determined by colorimetry.

Among the metabolic parameters, it is included:
plasma level of cholesterol in mmol/L, HDL-cholesterol in mmol/L, LDL-cholesterol in mmol/L, triglycerides in mmol/L, glucose (“glycemia”) in mmol/L, ferritin in µg/L, creatinine in µg/L (ICI), and vitamin D in ng/mL; these measurements are taken from fasting (12-hour) blood samples.

### Hormones

The following can be measured by liquid chromatography couple to the tandem mass spectrometry: LC-MS/MS

| | |
|---|---|
| DHEA: Dehydroepiandrosterone | 21DF: 21-Desoxycortisol |
| Cortisol | DOC: 11 Deoxycorticosterone |
| 16OHP: 16-hydroprogesterone | 11DF: 11-Deoxycortisol |
| Testosterone | 17OHP: 17-hydroxyprogesterone |
| 17OHP Pregn: 17 Hydroxy | 21DB: 21 desoxycorticosterone |
| Pregnenolone | DHT: Dehydrotestosterone |
| Corticosterone | Pregn: pregnenolone |
| Androstenedione | Progesterone |
| Cortisone | Aldosterone |
| 11BOH4: 11Beta-hydroxyDelta4Androstendione | |

The anti-mullerian hormone (AMH in ng/ml) can be quantified by enzyme-linked immunosorbent assay (ELISA).

Finally, another parameter that can be taken into account is the smoking status (also referred to as “CO exp”) which is assessed by the amount of exhaled carbon monoxide in ppm (Tabataba analyser-FIM medical, Villeurbanne 69625 France) [Deveci SE 2004 Respir Med] (Dupont, 2019 BACA).

For the implementation of the invention, these parameters are measured in the male and female of a fertile or idiopathic infertile couple, i.e. whose infertility cannot be explained by any of the anomalies described above.

There is no precise definition of idiopathic infertility; however, the couples who have failed to become pregnant after at least 12 months of unprotected sexual intercourse and in whom fertility tests are normal are considered to have idiopathic infertility: (i) no severe oligozoospermia, (ii) no alterations of the male reproductive organs such as undescended testicules, varicocele or infection, (iii) no female infertility factors such as anovulation, ovarian failure (based on follicle count and hormonal balance on day 3 (FSH, LH and estradiol)) or uterotubary pathology (assessed by hysterosalpingography).

Thus, in general, one or more of the eighty physiological parameters described above can be used, as will be explained in detail below, to provide an assessment of a risk of idiopathic infertility of the couple to be assessed. For example, all eighty parameters are used. However, this high number makes the procedure for obtaining measurements very heavy, impractical and expensive to implement. Thus, it is preferable to use only some (the most relevant) of these physiological parameters, for example less than twenty. One of the advantages of the invention is that the results obtained to classify the infertile couples is certainly relevant with 80 parameters, but it still remains relevant with a limited number of parameters, thus demonstrating the robustness of the system.

In the example described, the following thirteen physiological parameters are used:
Serum retinol levels in female;
Blood glucose in male and female;
Visceral fat content in female and male;
BMI for female and male;
Waist measurement for female and male;
Serum beta-carotene levels in male;
Serum lutein levels in female; and
Serum alpha carotene levels in male and female.

Thus, in the example described, the data receiving module 104 is designed to receive the measurements, noted p1...p13, of these thirteen physiological parameters respectively.

The physiological parameters form a multidimensional space (of dimension thirteen in the example described) of which each set of measurements is a point.

The system 100 further comprises a model 106 previously trained by machine learning to provide an idiopathic infertility score S of the couple to be assessed from the received measurements. Thus, in the example described, the model 106 is designed to provide the infertility score S from the thirteen measurements p1...p13 received.

Again in the example described, the model 106 is a linear model, so that the infertility score S is a linear combination of the received measurements p1...p13. In this linear combination, the measurements p1...p13 are respectively assigned to weights w1...w13, the weighted measurements being summed to obtain the infertility score S. Thus, in the example described, the score S is given by the following formula:

$$S = {\sum }_{n = 1}^{N} w n ⋅ p n + ε \qquad \text{[Math. 1]}$$

where N is the number of physiological parameters used, thirteen in the example described and ε is a constant representing a residue. This constant can be zero.

As will be explained in more detail later, the machine learning comprises the determination of the weights w1...w13 allowing the score S to be representative of the risk of idiopathic infertility of the couple to be assessed.

These weights w1...w13 can be updated by the update received by the receiving module 104.

Alternatively, the model 106 could comprise a neural network. In general, the model 106 implements a function including parameters and the machine learning of the model 106 comprises the determination of these parameters allowing the score S to be representative of the risk of idiopathic infertility of the couple to be assessed.

Preferably, the model 106 is previously trained so that the score S is within a predefined range, for example between 0 and 1 in the example described.

Thus, in general, the model 106 is a module for calculating the score S from the measurements received.

In the example described, the system 100 further comprises a module 108 for comparing the score S with a predefined threshold, for example 0.5 in the example described. The result of this comparison provides an assessment D of a medical risk of idiopathic infertility. For example, the risk assessment D is binary and indicates either a low risk of idiopathic infertility when the score S is above the predefined threshold or a high risk of idiopathic infertility when the score S is below the predefined threshold.

The system 100 further comprises, in the example described, a module 110 for projecting the set of measurements p1...p13 into a predefined projection plane of the multidimensional space. The resulting projection is denoted P.

The system 100 further comprises associations 112 associating treatments with respective predefined zones of the multidimensional space. For example, the proposed treatments comprise the artificial insemination and the in vitro fertilisation. In the example described, these zones are respectively defined by surfaces in the projection plane. Thus, a point in multidimensional space (i.e. a set of measurements) belongs to one of the predefined zones when its projection in the projection plane lies within the surface associated with that zone. Examples of zones will be described later, with reference to FIGS. 14 and 15.

The system 100 further comprises a module 114 for selecting the treatment T associated with the zone in which the measurements p1...p13 of the physiological parameters of the couple to be assessed are located.

The system 100 further comprises an information presentation module 116 designed to present to a user of the system 100 the score S and/or the risk assessment D. The presentation module 116 may further be designed to present the treatment T selected by the module 114 and/or the projection plane P with the position of the set of measurements in that projection plane.

The presentation module 116 is for example designed to cooperate with the human/machine interface 102. In the example described, the presentation module 116 is designed to cause information to be displayed on the touch screen.

In the example described, the system 100 is a computer system comprising a processing unit 118 (such as one or more microprocessors) and a main memory 120 coupled to the processing unit 118. In the example described, the system 100 further comprises a mass storage 122. A computer program 124 is stored in the mass storage 122 and contains computer program instructions. The computer program 124 is intended to be loaded into the main memory 120, so that the loaded instructions are executed by the processing unit 118.

Thus, the modules described above are implemented in the example described in the computer program as software modules.

For example, in particular where the system 100 is a smartphone, the computer program 124 may be in the form of an application (APP), for example available in the Apple (trademark) and/or Android (trademark) application shop.

Alternatively, some or all of the modules could be implemented as hardware modules, i.e. as an electronic circuit, for example micro-wired, not involving a computer program.

With reference to FIG. 2, an example 200 of an automated data treating method, within the scope of the example of the system 100 of FIG. 1, will now be described.

In a step 202, the receiving module 104 receives the measurements p1...p13.

In a step 204, the model 106 calculates the score S from the received measurements p1...p13.

In a step 206, the comparison module 108 compares the score S to the predefined threshold and determines the risk assessment D.

In parallel, in a step 208, the projection module 110 determines the projection P of the set of measurements p1...p13 in the projection plane.

In a step 210, the treatment selection module 114 selects the treatment T associated with the zone in which the measurements p1...p13 are located. In the example described, the module 114 determines the surface in which the projection P of the measurements p1...p13 is located and selects the treatment T associated with this surface.

In a step 212, the presentation module 116 receives the selected score S, risk assessment D, projection P and treatment T and cooperates with the human/machine interface 102 to display them.

For example, with reference to FIG. 3, the presentation module 116 causes a sentence to be displayed representing the risk assessment D, for example “high risk of idiopathic infertility” or “low risk of idiopathic infertility”, depending on the risk assessment D. Also for example, the presentation module 116 causes the score S and preferably the predefined threshold to be displayed. In addition, the presentation module 116 causes a name of the selected treatment T to be displayed, for example. In other embodiments, the presentation module 116 could cause the projection plane to be displayed with the projection P of the measurements p1...p13 of the couple to be assessed on it.

With reference to FIG. 4, a method 400 for obtaining a model for an automated system such as that of FIG. 1 will now be described.

In a step 402, for each of a plurality of couples in a developmental cohort, physiological parameter measurements of a male and a female of the couple are obtained, as well as a fertility status of that couple. The fertility status is binary and indicates either a fertility or an idiopathic infertility. For the method 400 to work, the fertility status of at least one of the couples indicates that the couple is idiopathic infertile and the fertility status of at least one other of the couples indicates that the couple is fertile. In some cases, some measurements may be missing. In addition, preferably a test cohort is also obtained.

In a step 404, the measurements of the development cohort are analysed for seeking statistically aberrant measurements for one or more couples in the development cohort. In addition, each couple the measurements of which are statistically aberrant is removed from the development cohort. To carry out this data analysis, the measurements are first centred and reduced. The Principal Component Analysis (PCA) can then be used, for example, to search for one or more statistically aberrant sets of measurements.

In the following of the method 400, the most relevant physiological parameters and a model using these relevant physiological parameters are searched for.

For this purpose, in a step 406, all the physiological parameters obtained are initially selected.

Then, in a step 408, a model is built. In the example described, this construction is iterative by reducing the number of physiological parameters used at each iteration. The step 408 comprises, for example, the following steps 408-2 to 408-8, which may thus be repeated several times.

In a step 408-2, an in-progress model is constructed by machine learning from measurements of the physiological parameters selected for the couples in the development cohort. The aim of machine learning is for the current model to provide a score S that reliably predicts the fertility status of couples in the development cohort and, subsequently, of a new couple whose measurements of the physiological parameter are provided to the model. In general, a training algorithm is used for this. The latter is designed to adjust parameters of the model from training data comprising input data and output data, so that the model provides, from known input data, known output data.

In the example described, the current model is a linear combination of the measurements of the selected physiological parameters and the machine learning is designed to determine weights of this linear combination, so that the linear combination allows to reliably predict the fertility statuses of the couples in the development cohort.

For example, an Orthogonal Partial Least Squares -Discriminant Analysis (OPLS-DA) is used.

Indeed, carrying out physiological parameter measurements on couples is a difficult operation to implement, so that measurements can generally only be obtained for a small number of couples, for example a few dozen to a few hundred. In addition, measurements may be missing. Furthermore, it is reasonable to assume that the physiological parameters are a priori multicollinear. Thus, the OPLS-DA analysis seems particularly appropriate as it is effective in the case of multicollinearity, even in a small cohort. Furthermore, it is robust, i.e. it can be carried out even with some missing measurements.

As is known per se, by noting X a matrix of measurements of the selected physiological parameters for the couples in the development cohort and Y a vector gathering the infertility status for these couples, the OPLS-DA analysis decomposes the matrices X and Y as follows:

$$X = T {P}^{T} + {E}_{X} \qquad \text{[Math. 2]}$$

$$Y = U {Q}^{T} + {E}_{Y}$$

where T and U are score matrices of the matrices X and Y respectively, P and Q are loadings matrices of the matrices X and Y respectively, and E_{X} and E_{Y} are respectively residuals, which the decomposition seeks to make as small as possible.

The weights of the linear combination, denoted wn with n = 1...N where N is the number of physiological parameters, are then the components of a vector W defined as follows:

$$T = X W * \qquad \text{[Math. 3]}$$

W* being the assistant vector (also referred to as transconjugate) of vector W.

At this stage, the current model is therefore the linear combination of the measurements of the physiological parameters, with the weights corresponding respectively to the components wn of the vector W.

In a step 408-4, the current model is assessed for validation or invalidation.

The assessment can be an internal assessment (i.e. based on the development cohort) and/or an external assessment (i.e. based on the test cohort).

The internal assessment may use, for example, one or more of the following: the coefficient of determination R², the permutation test, the prediction coefficient Q² and the average accuracy (i.e. the proportion of correct predictions made by the model).

The external assessment uses, for example, the receiver operating characteristic (ROC) of the test cohort. For example, an Area Under the Curve (AUC) of the ROC curve can be compared to a predefined absolute threshold or to a relative threshold, calculated from the AUC of the models obtained in the previous iterations. The relative threshold is for example a predefined percentage of the AUC of the model obtained in the previous iteration. The accuracy can also be used for the external assessment, based on the test cohort (test accuracy).

If the current model has not been validated in step 408-4, the last validated model is provided and the method proceeds to a step 412 which will be described later.

If the current model is validated in step 408-4, the method 400 continues with the following steps.

In a step 408-6, an importance in the predictions carried out by the current model of each selected physiological parameter is determined.

In the example described, the importance of each physiological parameter in the prediction is chosen as the Variable Importance in Projection (VIP), defined in the OPLS-DA analysis. By construction, the average of the VIP is equal to one.

Thus, the most important physiological parameters are those that have the greatest influence on the infertility score S. In other words, the least important physiological parameters are those which, if not taken into account in the model, would not change the infertility score S much.

In a step 408-8, a portion of the physiological parameters are selected according to their importance.

For example, the physiological parameters whose importance is above a predefined threshold are selected. In the example described, those physiological parameters whose VIP is greater than their mean (i.e. one in the example described) are selected.

The method 400 then returns to the step 408-2 with the physiological parameters selected in the step 408-8.

Preferably, in a step 412, a new model is defined, in the same way as in the step 408 (except for the assessment, which is only internal), but this time using the total cohort, i.e. the development and test cohorts. Thus, the step 412 comprises the steps 412-2 to 418-8 equivalent to the steps 408-2 to 408-8 respectively.

If the model obtained in the step 412 uses the same physiological parameters as the model obtained in the step 408, this is an indication that these physiological parameters are the most relevant. In this case, the model obtained in the step 412 is preferably implemented in the system 100.

With reference to FIGS. 5 to 12, an example of implementation of the method 400 will now be described.

### Step 402: Obtaining Measurements

In the example described, the development cohort comprises 136 couples, 73 of whom are idiopathic infertile and 63 fertile. The test cohort in the example described comprises 61 couples, 24 of whom are idiopathic infertile and 37 fertile. The ages of the individuals in the couples range from 18 to 38 years for female and 18 to 45 years for male. The idiopathic infertile couples have had idiopathic infertility for more than twelve months and the fertile couples are healthy, with a child under two years old and spontaneously conceived.

In addition, in the example described, the 80 physiological parameters detailed above are assessed on the couples of the developmental cohort and test cohort.

### Step 404: Statistical Checking of the Measurements

FIG. 5 shows the result of the principal component analysis on the 136 couples in the development cohort. As is apparent, the set of measurements of one of the couples (indicated by an arrow) is far away from the Hotelling ellipse E. This couple is therefore removed from the development cohort, which is thus reduced to 135 couples.

### Step 406: Initialization to All Physiological Parameters

In the example described, the 80 physiological parameters described above are initially selected.

The development cohort is then used to carry out the determination step 408 of a model.

### Steps 408-2 and 408-4: Obtaining and Validating a Model (Development Cohort, First Pass)

The model obtained from the development cohort (135 couples) is a linear combination of the 80 physiological parameters and is validated.

### Steps 408-6 and 408-8: Selection of Physiological Parameters (Developmental Cohort, First Pass)

The VIP of the 80 physiological parameters are determined and 24 of them are greater than one and therefore selected. These are:
Serum retinol levels in female;
Blood glucose in male and female;
Visceral fat content in female and male;
BMI for female and male;
Waist measurement for female and male;
Serum beta-carotene levels in male;
Serum lutein levels in female;
Serum alpha-carotene levels in male and female;
HDL in male;
Hip measurement of female and male;
CO exp of male;
Serum selenium levels in female;
Serum beta carotene levels in female;
Serum lutein levels in male;
Testosterone in male;
DHEA in male;
Cortisol in male; and
Androstenedione in male.

### Steps 408-2 and 408-4: Obtaining and Validating a Model (Development Cohort, Second Pass)

The model obtained from the development cohort (135 couples) is a linear combination of the 24 physiological parameters selected earlier and is validated.

### Steps 408-6 and 408-8: Selection of Physiological Parameters (Developmental Cohort, Second Pass)

The VIP of the 24 physiological parameters are determined and 13 of them are greater than one and therefore selected. These are the 13 physiological parameters already described earlier in the description of the system 100 in FIG. 1.

### Steps 408-2 and 408-4 (Developmental Cohort, Third Pass)

The model obtained from the development cohort (135 couples) is a linear combination of the 13 physiological parameters selected earlier and is validated.

Regarding the internal validation, in the example described, the R² of the model is 0.467 and the Q² of the model 0.410. The internal accuracy is 0.838. The validity of the model can also be appreciated by noticing that in FIG. 6, the fertile couples (triangles) are mainly in the right half of the graph, while idiopathic infertile couples (dots) are mainly in the left half of the graph.

Regarding the external validation, in the example described, the accuracy score is 0.705. This accuracy can also be appreciated by noticing that in FIG. 8, the fertile couples in the test cohort (green stars) are mainly found on the right half of the graph, while the idiopathic infertile couples in the test cohort (red stars) are mainly found on the left half of the graph.

### Steps 408-6 and 408-8: Selection of Physiological Parameters (Developmental Cohort, Third Pass)

The VIP of the 13 physiological parameters are determined. They are illustrated in FIG. 9. 5 of these are greater than one and therefore selected.

### Steps 408-2 and 408-4: Obtaining and Validating a Model (Development Cohort, Fourth Pass)

The model obtained (with the 5 selected parameters) from the development cohort (135 couples) is a linear combination of the 5 previously selected physiological parameters and this time it is not validated. For example, the AUC determined from the test cohort is below a predefined absolute threshold or relative to the AUC of the model of the previous pass.

Thus, the last validated model, i.e. the model of the third pass (using 13 physiological parameters), is provided at the end of the step 408.

The total cohort is then used to carry out the step 412 of determination of a model.

### Steps 412-2 and 412-4: Obtaining and Validating a Model (Total Cohort, First Pass)

The model obtained from the total cohort (196 couples) is a linear combination of the 80 physiological parameters and is validated.

### Steps 412-6 and 412-8: Selection of Physiological Parameters (Total Cohort, First Pass)

The VIP of 80 physiological parameters are determined. They are illustrated in FIG. 10. 27 of these are greater than one and comprise the 24 physiological parameters selected in the step 408-8 (first pass). Thus, these 24 physiological parameters are selected.

### Steps 412-2 and 412-4: Obtaining and Validating a Model (Total Cohort, Second Pass)

The model obtained from the total cohort (196 couples) is a linear combination of the 24 physiological parameters and is validated.

### Steps 412-6 and 412-8 Selection of Physiological Parameters (Total Cohort, Second Pass)

The VIP of the 24 physiological parameters are determined. They are shown in FIGS. 11 and 13 of them are greater than one and therefore selected. They are identical to those selected in the step 408-8 (second pass).

### Steps 412-2 and 412-4: Obtaining and Validating a Model (Total Cohort, Third Pass)

The model obtained from the total cohort (196 couples) is a linear combination of the 13 physiological parameters and is validated.

### Steps 412-6 and 412-8: Selection of Physiological Parameters (Total Cohort, Third Pass)

The VIP of the 13 physiological parameters are determined. They are shown in FIG. 12 and a number of them are greater than one and therefore selected.

### Steps 412-2 and 412-4: Obtaining and Validating a Model (Total Cohort, Fourth Pass)

The model obtained from the total cohort (196 couples) is a linear combination of the physiological parameters selected in the third pass and is not validated.

Thus, the model for the third pass (using 13 physiological parameters) is provided at the end of the step 412.

It will be appreciated that new couples can be added to the development cohort to refine the model. Thus, a new OPLS-DA analysis (as implemented for example in the steps of the method 400 detailed above) can be carried out with the development cohort thus enriched with new couples, in order to obtain updated weights for the model. These updated weights can then be transmitted to the systems 100 for updating their model 106.

It is clear that a system such as the one described above allow to reliably evaluate the risk of idiopathic infertility.

In addition to assessing the risk of idiopathic infertility, the method according to the present invention also allows to identify which of the physiological parameters considered in idiopathic infertile couples are most correlated with idiopathic infertility; this is illustrated in FIG. 13 in the above example.

Indeed, it is apparent from this FIG. 13 that, in the example described, the thirteen parameters are divided into three groups, with the physiological parameters in a same group being correlated with each other. The first group comprises the blood glucose in male and female, a second group comprises the visceral fat content, the body mass index and the waist circumference, both in male and female, and the third group comprises the serum retinol and lutein levels in female, the serum beta-carotene levels in male and the serum alpha-carotene levels in male and female.

Taking physiological parameters into account allows the practitioner to make recommendations to a couple with an idiopathic infertility; for example, still in the case of FIG. 13, an overweight or an abdominal obesity in the female, which is expressed by the parameters of quantity of visceral fat (“w_visceral fat”), BMI (“w_BMI”) and waist measurement (“w_waist measurement”), correlates with an infertility, the practitioner can then recommend a weight loss; again in this example, a high blood glucose in a male is correlated with an infertility, this can be taken into account to improve the diet and the lifestyle.

Of particular interest, this method can also highlight nutrient deficiencies or excesses in the male and/or female of couples assessed as idiopathic infertile.

The present invention thus also relates to a nutritional composition comprising one or more active compounds, the nature of which is obtained by the model obtaining method according to the invention, such as the method 400, which selects the physiological parameter or parameters for the last validated model that are most correlated with the idiopathic infertility and on which the active compound or compounds will have a beneficial effect.

According to an embodiment, this nutritional composition comprises at least one modulator (agonist or antagonist) allowing to correct a deficiency or an excess of at least one physiological parameter selected for the last validated model in the model obtaining method according to the invention, as implemented by the method 400; preferably, this nutritional composition is used for the treatment of the idiopathic infertility.

More particularly, this nutritional composition comprises at least one nutrient, a measurement of which forms a selected physiological parameter for the last validated model in the model obtaining method according to the invention, as implemented by the method 400; preferably, this nutritional composition is used for the treatment of the idiopathic infertility.

In the present case, the method according to the invention has shown that a deficiency of retinol, lutein, alpha-carotene and beta-carotene is associated with an idiopathic infertility.

Thus, the present invention further relates to:
a nutritional composition comprising at least one of the compounds selected from vitamin A, alpha-carotene, beta-carotene and lutein and a physiologically acceptable carrier for its use in treating idiopathic infertility; this nutritional composition may also comprise at least two, at least three or the four of the compounds;
a nutritional composition comprising at least one, at least two or the three compounds selected from vitamin A, alpha-carotene and lutein and a physiologically acceptable carrier for its use in treating the idiopathic infertility in a female
a nutritional composition comprising at least one or both of alpha-carotene and beta-carotene and a physiologically acceptable carrier for its use in treating the idiopathic infertility in a male.

The contents of the active compounds in the nutritional composition and its dosage will be adjusted according to the recommended daily intakes for each of the active compounds.

The nutritional compositions according to the invention can be administered by different routes; preferably, they are intended for oral administration and are advantageously formulated in the form of food or food supplements. Such formulations may comprise an ingestible support, the nature of which is adapted according to the type of composition under consideration, which may be selected from pills, soft gels or tablets, suspensions, oral supplements in dry form and oral supplements in liquid form.

The nutritional compositions may be prepared by any usual method known to the person skilled in the art to produce drinkable solutions, dragees, softgels, gels, emulsions, pills for swallowing or chewing, capsules, in particular soft or hard capsules, granules to be dissolved, syrups, solid or liquid foodstuffs and hydrogels allowing a controlled release, patches, food bars, powders, whether compacted or not, suspensions or liquid solutions, confectionery, fermented milk, fermented cheese, chewing gum, toothpaste or spray solutions.

The active compound or compounds of the nutritional compositions of the invention may furthermore be formulated with the usual excipients and components for such oral compositions or food supplements, namely fatty and/or aqueous components, humectants agents, thickening agents, preservatives agents, texture agents, flavour and/or coating agents, and/or antioxidant agents. The formulating agents and excipients for oral composition, and in particular for food supplements, are known in this field and are not described in detail here.

FIG. 14 shows the same graphs as in FIGS. 6 to 8 for three sets of physiological parameters: the 50 male physiological parameters (three graphs in the first row), the 30 female physiological parameters (three graphs in the second row) and the 80 physiological parameters (50 male physiological parameters and 30 female physiological parameters) (three graphs in the third row). Concerning the internal validation (graphs in the left-hand column), the R² of the models are respectively 0.471 (male), 0.560 (female) and 0.624 (male + female) and the Q2 are respectively 0.322 (male), 0.462 (female) and 0.487 (male + female). Concerning the external validation (middle and right column graphs), the accuracies are 0.590 (male), 0.574 (female) and 0.688 (male + female) respectively. Thus, it is apparent that taking into account both male and female parameters of the couple allows to improve the accuracy.

In FIG. 15, the triangles indicate the successful IVF and the circles indicate the successful inseminations. Conversely, in FIG. 16, the triangles indicate the IVF that did not result in pregnancy initiation and the circles indicate the inseminations that did not result in pregnancy initiation either. As is apparent, it seems that in the zone 1, the IVF has the best chance of success, while in the zone 2, the insemination seems to be sufficient to achieve a pregnancy. Thus, for example, it is possible to associate the zone 1 with the IVF treatment and the zone 2 with the insemination treatment, and to include these associations in the system 100, so that the system 100 proposes the associated treatment to the couple to be assessed whose set of measurements falls within one of these zones.

It should be noted that the invention is not limited to the embodiments described above. It will indeed appear to the person skilled in the art that various modifications can be made to the above-described embodiments, in the light of the teaching just disclosed.

In the foregoing detailed presentation of the invention, the terms used should not be interpreted as limiting the invention to the embodiments exposed in the present description, but should be interpreted to include all equivalents the anticipation of which is within the reach of the person skilled in the art by applying his general knowledge to the implementation of the teaching just disclosed.

1. An automated system for treating physiological parameter measurements for providing an assessment of a medical risk, the system comprising a model previously constructed by machine learning to provide an idiopathic infertility score for a couple to be assessed, formed of a male and a female, based on measurements received of physiological parameters, and wherein the physiological parameters comprise at least one physiological parameter of the male and at least one physiological parameter of the female.

2. The automated system of claim 1, wherein the model comprises a linear combination of the measurements of the physiological parameters.

3. The automated system of claim 1, wherein the physiological parameters comprise at least one of: an anthropometric parameter, a metabolic parameter and a parameter of oxidative status.

4. The automated system of claim 3, wherein the physiological parameters comprise the following variables:
Serum retinol levels in female;
Blood glucose in male and female;
Visceral fat content in female and male;
BMI for female and male;
Waist measurements for female and male;
Serum beta-carotene levels in male;
Serum lutein levels in female; and
Serum alpha carotene levels in male and female.

5. The automated system of claim 4, wherein the physiological parameters further comprise:
HDL in male;
Hip measurement of female and male;
CO exp of male;
Serum selenium levels in female;
Serum beta carotene levels in female;
Serum lutein levels in male;
Testosterone in male;
DHEA in male;
Cortisol in male; and
Androstenedione in male.

6. The automated system of claim 5, wherein the physiological parameters further comprise:
Weight of female and male;
HDL in female;
16OHP of male;
Serum retinol levels in male;
Corticosterone in male
21DB of male;
11 BOH4 of male;
17OHP of male;
Serum cobalamin levels in female and male;
Age of the female and the male;
Cortisone in male
21DF of male
11DF of male;
Serum alpha tocopherol levels in female and male;
DOC of male;
Triglycerides in male and female;
17OH Pregn of male;
Serum folate levels in female and male;
DHT in male;
Creatinine in male and female;
Pregn of male;
Serum lycopene levels in female and male;
Serum vitamin C levels in female;
Glutathione in male and female;
Vitamin D in female;
Systolic blood pressure in female and male;
Cholesterol in female and male;
LDL in female and male;
Progesterone in male;
Ferritin in female and male;
AMH in male;
Serum zinc levels in male and female;
Beta cryptoxanthin in female;
Diastolic blood pressure in female and male
Serum vitamin D levels in male;
Aldosterone in male;
Height of the male and the female;
Glutathione Peroxidase in male;
Selenium in male; and
Vitamin C in male.

7. The automated system according to claim 1, further comprising:
associations associating treatments with respective zones of a multidimensional space formed by the physiological parameters; and
a module for selecting the treatment associated with the zone in which the measurements of the physiological parameters of the couple to be assessed are located.

8. A method for obtaining a model for an automated system according to claim 1, comprising:
for each of a plurality of couples in a cohort, obtaining physiological parameter measurements from a male of the couple and a female of the couple and a fertility status of the couple, with the fertility status of at least one of the couples indicating that the couple is idiopathic infertile and the fertility status of at least one other of the couples indicating that the couple is fertile;
selecting any of the physiological parameters obtained; and
determining a model, comprising:
training a model by a training algorithm, based on measurements of selected physiological parameters of the couples in the cohort, so that the trained model provides a fertility score predicting the fertility status of the couple, with the training algorithm providing an importance of each physiological parameter in the prediction,
assessing the model to validate or invalidate the model,
if the model is validated, determining an importance of each physiological parameter in the prediction and selecting some of the physiological parameters according to their importance, and then repeating the step of determining a model with the newly selected physiological parameters,
if the model is invalidated, providing the last validated model.

9. The method of claim 8, further comprising, prior to the step of determining a model, identifying at least one couple whose measurements are statistically aberrant and removing each identified couple from the cohort.

10. A method for the automated treating of physiological parameter measurements for providing an assessment of a medical risk of idiopathic infertility, the method comprising using a model previously constructed by machine learning to provide a score of idiopathic infertility of a couple to be assessed, formed by a male and a female, based on received measurements of the physiological parameters, and wherein the physiological parameters comprise at least one physiological parameter of the male and at least one physiological parameter of the female.

11. The method according to claim 10, wherein the model comprises a linear combination of the measurements of the physiological parameters.

12. The method according to claim 10, wherein the physiological parameters comprise at least one of: an anthropometric parameter, a metabolic parameter and a parameter of the oxidative status.

13. The method according to claim 10, wherein zones of a multidimensional space formed by the physiological parameters are respectively associated with treatments and further comprising the selection of the treatment associated with the zone in which the measurements of the physiological parameters of the couple to be assessed are located.

14. A computer program downloadable from a communication network and/or recorded on a computer-readable medium, comprising instructions for executing a method according to claim 10, when said computer program is executed on a computer.

15. A method for assessing a medical risk of idiopathic infertility for a couple comprising the steps of:
collecting, for a couple consisting of a male and a female, at least one physiological parameter of the male and at least one physiological parameter of the female;
calculating an infertility score by inputting the parameters into an automated system for treating measurements of physiological parameters according to claim 1; and
comparing the score with a threshold, if the score is below the threshold, the couple will be assessed as being at risk of idiopathic infertility.

16. A nutritional composition comprising at least one modulator (agonist or antagonist) allowing for correcting a deficiency or an excess of a selected physiological parameter for the last validated model in the method according to claim 8.

17. A nutritional composition comprising at least one nutrient, a measurement of which forms a selected physiological parameter for the last validated model in the method according to claim 8.

18. The nutritional composition of claim 16, the composition being adapted for treatment of the idiopathic infertility.

19. A nutritional composition comprising at least one of the compounds selected from vitamin A, alpha-carotene, beta-carotene and lutein and a physiologically acceptable carrier for treating idiopathic infertility.

20. The nutritional composition according to claim 18, comprising at least two, compounds.

21. The nutritional composition according to claim 18, comprising at least one of the compounds selected from vitamin A, alpha-carotene and lutein and a physiologically acceptable carrier for treating the idiopathic infertility in a female.

22. A nutritional composition comprising at least one of the compounds selected from alpha-carotene and beta-carotene and a physiologically acceptable carrier for treating idiopathic infertility in a male.
